(19) United States
(12) Patent Application Publication
(10) Pub. No.: US 2022/0008130 A1
(43) Pub. Date: Jan. 13, 2022

(54) ACOUSTIC TISSUE IDENTIFICATION FOR BALLOON INTRAVASCULAR LITHOTRIPSY GUIDANCE

(71) Applicants: Boston Scientific Scimed, Inc., Maple Grove, MN (US); Bolt Medical, Inc., Carlsbad, CA (US)

(72) Inventors: Daniel Frank Massimini, Brooklyn Park, MN (US); Roger W. McGowan, Otsego, MN (US); Haiping Shao, Plymouth, MN (US); Darrin Dale Beekman, Osseo, MN (US); Eric Schultheis, San Clemente, CA (US)

(21) Appl. No.: 17/365,451

(22) Filed: Jul. 1, 2021

Related U.S. Application Data
(60) Provisional application No. 63/049,965, filed on Jul. 9, 2020.

(51) Int. Cl. A61B 18/26

(52) U.S. Cl. CPC A61B 18/26; A61B 2018/0022

(57) ABSTRACT

A catheter system (100) for treating a treatment site (106) within or adjacent to a vessel wall (208A) or a heart valve within a body (107) of a patient (109) includes an energy source (124), a balloon (104), an energy guide (122A), and a tissue identification system (142). The energy source (124) generates energy. The balloon (104) is positionable substantially adjacent to the treatment site (106). The balloon (104) includes a balloon wall (130) that defines a balloon interior (146). The balloon (104) can be configured to retain a balloon fluid (132) within the balloon interior (146). The energy guide (122A) is configured to receive energy from the energy source (124) and guide the energy into the balloon interior (146) so that plasma bubbles (134) are formed in the balloon fluid (132) within the balloon interior (146). The tissue identification system (142) can be configured to acoustically analyze tissue within the treatment site (106).

### RELATED APPLICATION

This application claims priority on U.S. Provisional Application Ser. No. 63/049,965, filed on Jul. 9, 2020. To the extent permitted, the contents of U.S. Provisional Application Ser. No. 63/049,965 are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions, such as severely calcified vascular lesions, can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

Intravascular lithotripsy is one method that has been recently used with some success for breaking up vascular lesions within vessels in the body. Intravascular lithotripsy utilizes a combination of pressure waves and bubble dynamics that are generated intravascularly in a fluid-filled balloon catheter. In particular, during a intravascular lithotripsy treatment, a high energy source is used to generate plasma and ultimately pressure waves as well as a rapid bubble expansion within a fluid-filled balloon to crack calcification at a lesion site within the vasculature. The associated rapid bubble formation from the plasma initiation and resulting localized fluid velocity within the balloon transfers mechanical energy though the incompressible fluid to impart a fracture force on the intravascular calcium, which is opposed to the balloon wall. The rapid change in fluid momentum upon hitting the balloon wall is known as hydraulic shock, or water hammer.

There is an ongoing desire to enhance vessel patency and optimization of therapy delivery parameters within a intravascular lithotripsy catheter system.

### SUMMARY

The present invention is directed toward a catheter system for treating a treatment site within or adjacent to the vessel wall or a heart valve within a body of a patient. In various embodiments, the catheter system includes an energy source, a balloon, an energy guide, and a tissue identification system. The energy source generates energy. The balloon is positionable substantially adjacent to the treatment site. The balloon includes a balloon wall that defines a balloon interior. The balloon can be configured to retain a balloon fluid within the balloon interior. The energy guide is configured to receive energy from the energy source and guide the energy into the balloon interior so that plasma is formed in the balloon fluid within the balloon interior. The tissue identification system can be configured to acoustically analyze tissue within the treatment site.

In some embodiments, the tissue identification system is configured to utilize acoustic tissue identification to provide real-time feedback regarding tissue type and quantity within the treatment site.

In certain embodiments, the catheter system further includes a plasma generator that is positioned at a guide distal end of the energy guide, the plasma generator being configured to generate the plasma in the balloon fluid within the balloon interior. In such embodiments, the plasma formation causes rapid bubble formation and imparts pressure waves upon the balloon wall adjacent to the treatment site.

In some embodiments, the energy source generates pulses of energy that are guided along the energy guide into the balloon interior to induce the plasma formation in the balloon fluid within the balloon interior.

In one embodiment, the energy source is a laser source that provides pulses of laser energy.

In certain embodiments, the energy guide can include an optical fiber. In one embodiment, the energy source is a high voltage energy source that provides pulses of high voltage.

In various embodiments, the energy guide can include an electrode pair including spaced apart electrodes that extend into the balloon interior; and pulses of high voltage from the energy source can be applied to the electrodes and form an electrical arc across the electrodes.

In some embodiments, the tissue identification system includes an identification energy source that generates energy, and an acoustic source that receives the energy from the identification energy source in the form of an identification source beam and converts the identification source beam into acoustic energy that is directed toward the tissue within the treatment site.

In one embodiment, the tissue identification system is an ultrasound system, and the identification energy source includes a pulse echo generator.

In certain embodiments, the identification energy source can include a light source such as a laser.

In various embodiments, the acoustic source can include a piezoelectric transducer.

In some embodiments, the acoustic source can include a photoacoustic transducer.

In some embodiments, the tissue identification system further includes an identification energy guide that guides the identification source beam from the identification energy source into the balloon interior.

In certain embodiments, the acoustic source can be coupled to the identification energy guide.

In some embodiments, the identification energy guide includes a diverter that is coupled to a guide distal end of the identification energy guide to direct the acoustic energy toward the treatment site.

In certain embodiments, the tissue identification system further includes an acoustic detector that is configured to detect acoustic energy within the balloon interior.

In some embodiments, at least a portion of the acoustic energy directed toward the treatment site can be reflected by the tissue within the treatment site and can be directed toward the acoustic detector.

In some embodiments, the acoustic detector is coupled to a guide distal end of a second identification energy guide.

In certain embodiments, the acoustic detector can be positioned outside the body of the patient.

In one embodiment, the acoustic detector is positioned adjacent to the body of the patient.

In certain embodiments, the acoustic detector includes a piezoelectric transducer.

In some embodiment, the acoustic source and the acoustic detector can both be encompassed within a single piezoelectric transducer.

In various embodiments, the acoustic source is a piezoelectric transducer that is coupled to a first identification energy guide, and the acoustic detector is a piezoelectric transducer that is coupled to a second identification energy guide that is different than the first identification energy guide.

In certain embodiments, the acoustic detector includes a Fabry-Perot cavity that is coupled to the guide distal end of the second identification energy guide.

In various embodiments, the acoustic detector generates a detector signal based on the detected acoustic energy within the balloon interior and sends the detector signal to control electronics.

In some embodiments, the control electronics analyze the detector signal to determine the tissue type and quantity within the treatment site.

In various embodiments, the acoustic detector is electrically coupled to the control electronics via a wired connection.

In some embodiments, the acoustic detector is electrically coupled to the control electronics via a wireless connection.

The present invention is also directed toward a method for treating a treatment site within or adjacent to a vessel wall or heart valve utilizing any of the catheter systems described herein.

The present invention is also directed toward a catheter system for treating a treatment site within or adjacent to a vessel wall or heart valve within a body of a patient, the catheter system including a light source that generates light energy; a balloon that is positionable substantially adjacent to the treatment site, the balloon having a balloon wall that defines a balloon interior, the balloon being configured to retain a balloon fluid within the balloon interior; a light guide that is configured to receive light energy from the light source and guide the light energy into the balloon interior so that plasma is formed in the balloon fluid within the balloon interior; and a tissue identification system that is configured to acoustically analyze tissue within the treatment site.

The present invention is also directed toward a method for treating a treatment site within or adjacent to a vessel wall or a heart valve within a body of a patient, the method including the steps of generating energy with an energy source; positioning a balloon substantially adjacent to the treatment site, the balloon having a balloon wall that defines a balloon interior; retaining a balloon fluid within the balloon interior; receiving energy from the energy source with an energy guide; guiding the energy with the energy guide into the balloon interior so that plasma is formed in the balloon fluid within the balloon interior; and acoustically analyzing tissue within the treatment site with a tissue identification system.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic cross-sectional view of an embodiment of a catheter system in accordance with various embodiments herein, the catheter system including a tissue identification system having features of the present invention;

FIG. 2 is a simplified schematic view of a portion of an embodiment of the catheter system including an embodiment of the tissue identification system;

FIG. 3 is a simplified schematic view of a portion of another embodiment of the catheter system including another embodiment of the tissue identification system;

FIG. 4 is a simplified schematic view of a portion of still another embodiment of the catheter system including still another embodiment of the tissue identification system;

FIG. 5 is a simplified schematic view of a portion of yet another embodiment of the catheter system including yet another embodiment of the tissue identification system; and

FIG. 6 is a simplified schematic view of a portion of still yet another embodiment of the catheter system including still yet another embodiment of the tissue identification system.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions (also sometimes referred to herein as “treatment sites”) can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

The catheter systems and related methods disclosed herein are configured to enhance the intravascular lithotripsy therapeutic outcome by providing real-time feedback on vessel patency and optimization of the therapy delivery parameters. More specifically, the catheter systems and related methods disclosed herein include a feedback mechanism in the form of an acoustic tissue identification system that provides details on tissue type, quantity and location at a particular treatment site. As provided herein, different acoustic tissue identification methodologies can be utilized within the acoustic tissue identification system for purposes of providing the desired real-time feedback on vessel patency and optimization of the therapy delivery parameters. For example, acoustic tissue identification methods such as intravascular ultrasound (IVUS) are available commercially, but have never been combined with a intravascular lithotripsy catheter. Such an IVUS system can be specifically tailored to identify calcium or partial calcium within the analyzed tissue. Alternatively, the acoustic tissue identification system can employ an all-optical acoustic system. It is appreciated that such acoustic methods are typically accurate and fast, and thus can be utilized effectively with generally unperceivable additional procedure time.

As described herein, in certain embodiments, utilizing an acoustic source such as a piezoelectric transducer or a photoacoustic transducer, and/or an acoustic detector such as a piezoelectric transducer or a Fabry-Perot cavity on an additional fiber optic, the intravascular lithotripsy catheter system utilizing such a tissue identification system that employs acoustic tissue identification can optimize treatment location and duration, energy and frequency, as well as provide therapy verification in real-time. Ultimately, a smart intravascular lithotripsy device with acoustic sensing, such as described herein, can improve patient outcomes while minimizing collateral damage to surrounding tissues.

In various embodiments, the catheter systems and related methods of the present invention utilize an energy source, e.g., in some embodiments, a light source such as a laser source or another suitable energy source, which provides energy that is guided by an energy guide, e.g., in some embodiments, a light guide or another suitable energy guide, to create a localized plasma in the balloon fluid that is retained within a balloon interior of an inflatable balloon of the catheter. As such, the energy guide can sometimes be referred to as, or can be said to incorporate a “plasma generator” at or near a guide distal end of the energy guide that is positioned within the balloon interior. The creation of the localized plasma, in turn, induces a high energy bubble inside the balloon interior to create pressure waves and/or pressure waves to impart pressure onto and induce fractures in a treatment site, such as a calcified vascular lesion or a fibrous vascular lesion, at a treatment site within or adjacent to a blood vessel wall or heart valve within a body of a patient.

As described in detail herein, the catheter systems of the present invention include and/or incorporate an acoustic tissue identification system that is specifically configured to provide real-time feedback on tissue type, quantity and location as a means to enhance vessel patency and optimization of the therapy delivery parameters. As described in various embodiments, the tissue identification system can provide advantages such as (i) tissue identification at the therapy location site provides an opportunity to optimize therapy parameters with the prospect of improved vessel patency, (ii) the acoustic tissue identification methods are accurate and fast, with no additional procedure time required, (iii) the tissue identification as well as the actual treatment performed at the therapy location can be performed in a single-use device, thereby simplifying the overall process of treatment and monitoring of efficacy of the treatment, and (iv) the tissue identification system can truly provide acoustic monitoring of progression of the procedure and efficacy of treatment in real-time.

In various embodiments, the catheter systems can include a catheter configured to advance to the vascular lesion located at the treatment site within or adjacent a blood vessel within the body of the patient. The catheter includes a catheter shaft, and a balloon that is coupled and/or secured to the catheter shaft. The balloons herein can include a balloon wall that defines a balloon interior. The balloons can be configured to receive the balloon fluid within the balloon interior to expand from a deflated configuration suitable for advancing the catheter through a patient's vasculature, to an inflated configuration suitable for anchoring the catheter in position relative to the treatment site. The catheter systems also include one or more energy guides disposed along the catheter shaft and within the balloon. Each energy guide can be configured for generating pressure waves within the balloon for disrupting the treatment sites. The catheter systems utilize energy from an energy source to generate the plasma, i.e. via the plasma generator, within the balloon fluid at or near a guide distal end of the energy guide disposed within the balloon interior of the balloon located at the treatment site. The plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid retained within the balloon and thereby impart pressure waves upon the treatment site. In some embodiments, the energy source can be configured to provide sub-millisecond pulses of energy from the energy source to initiate plasma formation in the balloon fluid within the balloon to cause rapid bubble formation and to impart pressure waves upon the balloon wall at the treatment site. Thus, the pressure waves can transfer mechanical energy through an incompressible balloon fluid to the treatment site to impart a fracture force on the intravascular lesion.

As used herein, the terms “intravascular lesion”, “vascular lesion” and “treatment site” are used interchangeably unless otherwise noted. As such, the intravascular lesions and/or the vascular lesions are sometimes referred to herein simply as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

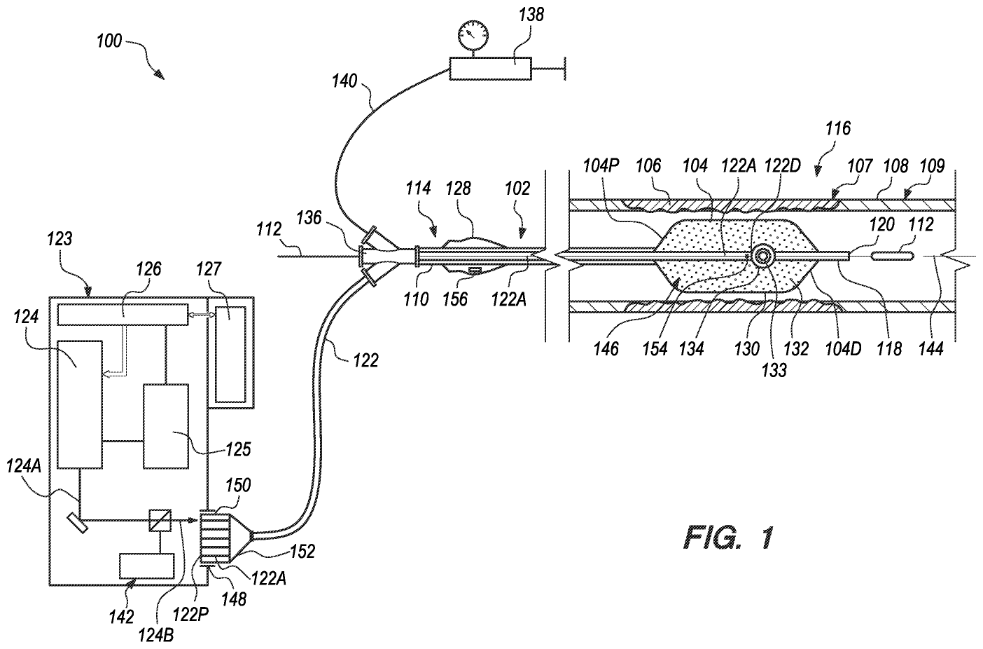

It is appreciated that the catheter systems disclosed herein can include many different forms. Referring now to FIG. 1, a schematic cross-sectional view is shown of a catheter system 100 in accordance with various embodiments herein. As described herein, the catheter system 100 is suitable for imparting pressure to induce fractures in one or more treatment sites within or adjacent a vessel wall of a blood vessel or a heart valve. In the embodiment illustrated in FIG. 1, the catheter system 100 can include one or more of a catheter 102, a light guide bundle 122 including one or more light guides 122A, a source manifold 136, a fluid pump 138, a system console 123 including one or more of a light source 124, a power source 125, a system controller 126, and a graphic user interface 127 (a “GUI”), a handle assembly 128, and an acoustic tissue identification system 142 (also referred to herein more simply as a “tissue identification system”). Alternatively, the catheter system 100 can have more components or fewer components than those specifically illustrated and described in relation to FIG. 1.

The catheter 102 is configured to move to a treatment site 106 within or adjacent to a blood vessel 108 or heart valve within a body 107 of a patient 109. The catheter 102 can include an inflatable balloon 104 (sometimes referred to herein simply as a “balloon”), a catheter shaft 110 and a guidewire 112. The balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter shaft 110 can also include a guidewire lumen 118 which is configured to move over the guidewire 112. As utilized herein, the guidewire lumen 118 is intended to define the structure that provides a conduit through which the guidewire extends. The catheter shaft 110 can further include an inflation lumen (not shown). In some embodiments, the catheter 102 can have a distal end opening 120 and can accommodate and be tracked over the guidewire 112 as the catheter 102 is moved and positioned at or near the treatment site 106.

As described in detail herein, the tissue identification system 142 is configured to provide real-time feedback on tissue type, quantity and location in order to effectively enhance vessel patency and optimization of therapy delivery parameters. More particularly, the tissue identification system 142 is configured to utilize acoustic sensing capabilities in order to improve patient outcomes while minimizing collateral damage to surrounding tissues.

The catheter shaft 110 of the catheter 102 can be coupled to the one or more light guides 122A of the light guide bundle 122 that are in optical communication with the light source 124. The light guide(s) 122A can be disposed along the catheter shaft 110 and within the balloon 104. In some embodiments, each light guide 122A can be an optical fiber and the light source 124 can be a laser. The light source 124 can be in optical communication with the light guides 122A at the proximal portion 114 of the catheter system 100.

In some embodiments, the catheter shaft 110 can be coupled to multiple light guides 122A such as a first light guide, a second light guide, a third light guide, etc., which can be disposed at any suitable positions about the guidewire lumen 118 and/or the catheter shaft 110. For example, in certain non-exclusive embodiments, two light guides 122A can be spaced apart by approximately 180 degrees about the circumference of the guidewire lumen 118 and/or the catheter shaft 110; three light guides 122A can be spaced apart by approximately 120 degrees about the circumference of the guidewire lumen 118 and/or the catheter shaft 110; or four light guides 122A can be spaced apart by approximately 90 degrees about the circumference of the guidewire lumen 118 and/or the catheter shaft 110. Still alternatively, multiple light guides 122A need not be uniformly spaced apart from one another about the circumference of the guidewire lumen 118 and/or the catheter shaft 110. More particularly, it is further appreciated that the light guides 122A described herein can be disposed uniformly or non-uniformly about the guidewire lumen 118 and/or the catheter shaft 110 to achieve the desired effect in the desired locations.

The balloon 104 can include a balloon wall 130 that defines a balloon interior 146, and can be inflated with a balloon fluid 132 to expand from a deflated configuration suitable for advancing the catheter 102 through a patient's vasculature, to an inflated configuration suitable for anchoring the catheter 102 in position relative to the treatment site 106. Stated in another manner, when the balloon 104 is in the inflated configuration, the balloon wall 130 of the balloon 104 is configured to be positioned substantially adjacent to the treatment site 106. It is appreciated that although FIG. 1 illustrates the balloon wall 130 of the balloon 104 being shown spaced apart from the treatment site 106 of the blood vessel 108, this is done merely for ease of illustration, and the balloon wall 130 of the balloon 104 will typically be substantially directly adjacent to the treatment site 106 when the balloon is in the inflated configuration.

In some embodiments, the light source 124 of the catheter system 100 can be configured to provide sub-millisecond pulses of light from the light source 124, along the light guides 122A, to a location within the balloon interior 146 of the balloon 104, thereby inducing plasma formation in the balloon fluid 132 within the balloon interior 146 of the balloon 104, i.e. via a plasma generator 133 located at a guide distal end 122D of the light guide 122A. The plasma formation causes rapid bubble formation, and imparts pressure waves upon the treatment site 106. Exemplary plasma-induced bubbles are shown as bubbles 134 in FIG. 1.

It is appreciated that although the catheter systems 100 illustrated herein are generally described as including a light source 124 and one or more light guides 122A, the catheter system 100 can alternatively include any suitable energy source and energy guides for purposes of generating the desired plasma in the balloon fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the pressure waves within the balloon fluid 132 that are utilized to provide the fracture force onto the vascular lesions at the treatment site 106. Still alternatively, the energy source 124 and/or the energy guides 122A can have another suitable design and/or configuration.

The balloons 104 suitable for use in the catheter systems 100 described in detail herein include those that can be passed through the vasculature of a patient when in the deflated configuration. In some embodiments, the balloons 104 herein are made from silicone. In other embodiments, the balloons 104 herein are made from polydimethylsiloxane (PDMS), polyurethane, polymers such as PEBAX™ material available from Arkema, which has a location at King of Prussia, Pa., USA, nylon, and the like. In some embodiments, the balloons 104 can include those having diameters ranging from one millimeter (mm) to 25 mm in diameter. In some embodiments, the balloons 104 can include those having diameters ranging from at least 1.5 mm to 14 mm in diameter. In some embodiments, the balloons 104 can include those having diameters ranging from at least one mm to five mm in diameter.

Additionally, in some embodiments, the balloons 104 herein can include those having a length ranging from at least three mm to 300 mm. More particularly, in some embodiments, the balloons 104 herein can include those having a length ranging from at least eight mm to 200 mm. It is appreciated that balloons 104 of greater length can be positioned adjacent to larger treatment sites 106, and, thus, may be usable for imparting pressure onto and inducing fractures in larger vascular lesions or multiple vascular lesions at precise locations within the treatment site 106. It is further appreciated that such longer balloons 104 can also be positioned adjacent to multiple treatment sites 106 at any given time.

Further, the balloons 104 herein can be inflated to inflation pressures of between approximately one atmosphere (atm) and 70 atm. In some embodiments, the balloons 104 herein can be inflated to inflation pressures of from at least 20 atm to 70 atm. In other embodiments, the balloons 104 herein can be inflated to inflation pressures of from at least six atm to 20 atm. In still other embodiments, the balloons 104 herein can be inflated to inflation pressures of from at least three atm to 20 atm. In yet other embodiments, the balloons 104 herein can be inflated to inflation pressures of from at least two atm to ten atm.

Still further, the balloons 104 herein can include those having various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloons 104 herein can include a drug eluting coating or a drug eluting stent structure. The drug elution coating or drug eluting stent can include one or more therapeutic agents including anti-inflammatory agents, anti-neoplastic agents, anti-angiogenic agents, and the like.

The balloon fluid 132 can be a liquid or a gas. Exemplary balloon fluids 132 suitable for use herein can include, but are not limited to one or more of water, saline, contrast medium, fluorocarbons, perfluorocarbons, gases, such as carbon dioxide, and the like. In some embodiments, the balloon fluids 132 described can be used as base inflation fluids. In some embodiments, the balloon fluids 132 include a mixture of saline to contrast medium in a volume ratio of 50:50. In other embodiments, the balloon fluids 132 include a mixture of saline to contrast medium in a volume ratio of 25:75. In still other embodiments, the balloon fluids 132 include a mixture of saline to contrast medium in a volume ratio of 75:25. Additionally, the balloon fluids 132 suitable for use herein can be tailored on the basis of composition, viscosity, and the like in order to manipulate the rate of travel of the pressure waves therein. In certain embodiments, the balloon fluids 132 suitable for use herein are biocompatible. A volume of balloon fluid 132 can be tailored by the chosen light source 124 and the type of balloon fluid 132 used.

In some embodiments, the contrast agents used in the contrast media herein can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C5F12).

Additionally, the balloon fluids 132 herein can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 μm) of the electromagnetic spectrum. Suitable absorptive agents can include those with absorption maxima along the spectrum from at least ten nm to 2.5 μm. Alternatively, the balloon fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers described herein can include neodymium:yttrium-aluminum-garnet (Nd:YAG−emission maximum=1064 nm) lasers, holmium:YAG (Ho:YAG−emission maximum=2.1 μm) lasers, or erbium:YAG (Er:YAG−emission maximum=2.94 μm) lasers. In some embodiments, the absorptive agents used herein can be water soluble. In other embodiments, the absorptive agents used herein are not water soluble. In some embodiments, the absorptive agents used in the balloon fluids 132 herein can be tailored to match the peak emission of the light source 124. Various light sources 124 having emission wavelengths of at least ten nanometers to one millimeter are discussed elsewhere herein.

It is appreciated that the catheter system 100 and/or the light guide bundle 122 disclosed herein can include any number of light guides 122A in optical communication with the light source 124 at the proximal portion 114, and with the balloon fluid 132 within the balloon interior 146 of the balloon 104 at the distal portion 116. For example, in some embodiments, the catheter system 100 and/or the light guide bundle 122 can include from one light guide 122A to five light guides 122A. In other embodiments, the catheter system 100 and/or the light guide bundle 122 can include from five light guides 122A to fifteen light guides 122A. In yet other embodiments, the catheter system 100 and/or the light guide bundle 122 can include from ten light guides 122A to thirty light guides 122A. Alternatively, in still other embodiments, the catheter system 100 and/or the light guide bundle 122 can include greater than thirty light guides 122A.

As noted above, the energy guides 122A can have any suitable design for purposes of generating plasma and/or pressure waves in the balloon fluid 132 within the balloon interior 146. Thus, the particular description of the light guides 122A herein is not intended to be limiting in any manner, except for as set forth in the claims appended hereto.

In certain embodiments, the light guides 122A herein can include an optical fiber or flexible light pipe. The light guides 122A herein can be thin and flexible and can allow light signals to be sent with very little loss of strength. The light guides 122A herein can include a core surrounded by a cladding about its circumference. In some embodiments, the core can be a cylindrical core or a partially cylindrical core. The core and cladding of the light guides 122A can be formed from one or more materials, including but not limited to one or more types of glass, silica, or one or more polymers. The light guides 122A may also include a protective coating, such as a polymer. It is appreciated that the index of refraction of the core will be greater than the index of refraction of the cladding.

Each light guide 122A can guide light along its length from a proximal portion, i.e. a guide proximal end 122P, to a distal portion, i.e. the guide distal end 122D, having at least one optical window (not shown) that is positioned within the balloon interior 146. The light guides 122A can create a light path as a portion of an optical network including the light source 124. The light path within the optical network allows light to travel from one part of the network to another. Both the optical fiber and the flexible light pipe can provide a light path within the optical networks herein.

Further, the light guides 122A herein can assume many configurations about and/or relative to the catheter shaft 110 of the catheters 102 described herein. In some embodiments, the light guides 122A can run parallel to the longitudinal axis 144 of the catheter shaft 110. In some embodiments, the light guides 122A can be physically coupled to the catheter shaft 110. In other embodiments, the light guides 122A can be disposed along a length of an outer diameter of the catheter shaft 110. In yet other embodiments, the light guides 122A herein can be disposed within one or more light guide lumens within the catheter shaft 110.

Additionally, it is further appreciated that the light guides 122A can be disposed at any suitable positions about the circumference of the guidewire lumen 118 and/or the catheter shaft 110, and the guide distal end 122D of each of the light guides 122A can be disposed at any suitable longitudinal position relative to the length of the balloon 104 and/or relative to the length of the guidewire lumen 118.

Further, the light guides 122A herein can include one or more photoacoustic transducers 154, where each photoacoustic transducer 154 can be in optical communication with the light guide 122A within which it is disposed. In some embodiments, the photoacoustic transducers 154 can be in optical communication with the guide distal end 122D of the light guide 122A. Additionally, in such embodiments, the photoacoustic transducers 154 can have a shape that corresponds with and/or conforms to the guide distal end 122D of the light guide 122A.

The photoacoustic transducer 154 is configured to convert light energy into an acoustic wave at or near the guide distal end 122D of the light guide 122A. It is appreciated that the direction of the acoustic wave can be tailored by changing an angle of the guide distal end 122D of the light guide 122A.

It is further appreciated that the photoacoustic transducers 154 disposed at the guide distal end 122D of the light guide 122A herein can assume the same shape as the guide distal end 122D of the light guide 122A. For example, in certain non-exclusive embodiments, the photoacoustic transducer 154 and/or the guide distal end 122D can have a conical shape, a convex shape, a concave shape, a bulbous shape, a square shape, a stepped shape, a half-circle shape, an ovoid shape, and the like. It is also appreciated that the light guide 122A can further include additional photoacoustic transducers 154 disposed along one or more side surfaces of the length of the light guide 122A.

The light guides 122A described herein can further include one or more diverting features or “diverters” (not shown in FIG. 1) within the light guide 122A that are configured to direct light to exit the light guide 122A toward a side surface e.g., at or near the guide distal end 122D of the light guide 122A, and toward the balloon wall 130. A diverting feature can include any feature of the system herein that diverts light from the light guide 122A away from its axial path toward a side surface of the light guide 122A. Additionally, the light guides 122A can each include one or more light windows disposed along the longitudinal or circumferential surfaces of each light guide 122A and in optical communication with a diverting feature. Stated in another manner, the diverting features herein can be configured to direct light in the light guide 122A toward a side surface, e.g., at or near the guide distal end 122D, where the side surface is in optical communication with a light window. The light windows can include a portion of the light guide 122A that allows light to exit the light guide 122A from within the light guide 122A, such as a portion of the light guide 122A lacking a cladding material on or about the light guide 122A.

Examples of the diverting features suitable for use herein include a reflecting element, a refracting element, and a fiber diffuser. Additionally, the diverting features suitable for focusing light away from the tip of the light guides 122A herein can include, but are not to be limited to, those having a convex surface, a gradient-index (GRIN) lens, and a mirror focus lens. Upon contact with the diverting feature, the light is diverted within the light guide 122A to either a plasma generator 133 or the photoacoustic transducer 154 that is in optical communication with a side surface of the light guide 122A. As noted, the photoacoustic transducer 154 then converts light energy into an acoustic wave that extends away from the side surface of the light guide 122A.

The source manifold 136 can be positioned at or near the proximal portion 114 of the catheter system 100. The source manifold 136 can include one or more proximal end openings that can receive the plurality of light guides 122A of the light guide bundle 122, the guidewire 112, and/or an inflation conduit 140 that is coupled in fluid communication with the fluid pump 138. The catheter system 100 can also include the fluid pump 138 that is configured to inflate the balloon 104 with the balloon fluid 132, i.e. via the inflation conduit 140, as needed.

As noted above, in the embodiment illustrated in FIG. 1, the system console 123 includes one or more of the light source 124, the power source 125, the system controller 126, and the GUI 127. Alternatively, the system console 123 can include more components or fewer components than those specifically illustrated in FIG. 1. For example, in certain non-exclusive alternative embodiments, the system console 123 can be designed without the GUI 127. Still alternatively, one or more of the light source 124, the power source 125, the system controller 126, and the GUI 127 can be provided within the catheter system 100 without the specific need for the system console 123.

Further, as illustrated in FIG. 1, in certain embodiments, at least a portion of the tissue identification system 142 can also be positioned substantially within the system console 123. Alternatively, components of the tissue identification system 142 can be positioned in a different manner than what is specifically shown in FIG. 1.

Additionally, as shown, the system console 123, and the components included therewith, is operatively coupled to the catheter 102, the light guide bundle 122, and the remainder of the catheter system 100. For example, in some embodiments, as illustrated in FIG. 1, the system console 123 can include a console connection aperture 148 (also sometimes referred to generally as a “socket”) by which the light guide bundle 122 is mechanically coupled to the system console 123. In such embodiments, the light guide bundle 122 can include a guide coupling housing 150 (also sometimes referred to generally as a “ferrule”) that houses a portion, e.g., the guide proximal end 122P, of each of the light guides 122A. The guide coupling housing 150 is configured to fit and be selectively retained within the console connection aperture 148 to provide the desired mechanical coupling between the light guide bundle 122 and the system console 123.

Further, the light guide bundle 122 can also include a guide bundler 152 (or “shell”) that brings each of the individual light guides 122A closer together so that the light guides 122A and/or the light guide bundle 122 can be in a more compact form as it extends with the catheter 102 into the blood vessel 108 during use of the catheter system 100.

As provided herein, the light source 124 can be selectively and/or alternatively coupled in optical communication with each of the light guides 122A, i.e. to the guide proximal end 122P of each of the light guides 122A, in the light guide bundle 122. In particular, the light source 124 is configured to generate light energy in the form of a source beam 124A, e.g., a pulsed source beam, that can be selectively and/or alternatively directed to and received by each of the light guides 122A in the light guide bundle 122 as an individual guide beam 1248. Alternatively, the catheter system 100 can include more than one light source 124. For example, in one non-exclusive alternative embodiment, the catheter system 100 can include a separate light source 124 for each of the light guides 122A in the light guide bundle 122.

The light source 124 can have any suitable design. In certain embodiments, as noted above, the light source 124 can be configured to provide sub-millisecond pulses of light from the light source 124 that are focused onto a small spot in order to couple it into the guide proximal end 122P of the light guide 122A. Such pulses of light energy are then directed along the light guides 122A to a location within the balloon 104, thereby inducing plasma formation in the balloon fluid 132 within the balloon interior 146 of the balloon 104. In particular, the light energy emitted at the guide distal end 122D of the light guide 122A energizes the plasma generator 133 to form the plasma within the balloon fluid 132 within the balloon interior 146. The plasma formation causes rapid bubble formation, and imparts pressure waves upon the treatment site 106. In such embodiments, the sub-millisecond pulses of light from the light source 124 can be delivered to the treatment site 106 at a frequency of between approximately one hertz (Hz) and 5000 Hz. In some embodiments, the sub-millisecond pulses of light from the light source 124 can be delivered to the treatment site 106 at a frequency of between approximately 30 Hz and 1000 Hz. In other embodiments, the sub-millisecond pulses of light from the light source 124 can be delivered to the treatment site 106 at a frequency of between approximately ten Hz and 100 Hz. In yet other embodiments, the sub-millisecond pulses of light from the light source 124 can be delivered to the treatment site 106 at a frequency of between approximately one Hz and 30 Hz. Alternatively, the sub-millisecond pulses of light can be delivered to the treatment site 106 at a frequency that can be greater than 5000 Hz.

It is appreciated that although the light source 124 is typically utilized to provide pulses of light energy, the light source 124 can still be described as providing a single source beam 124A, i.e. a single pulsed source beam.

The light sources 124 suitable for use herein can include various types of light sources including lasers and lamps. Alternatively, as noted above, the light sources 124, as referred to herein, can include any suitable type of energy source.

Suitable lasers can include short pulse lasers on the sub-millisecond timescale. In some embodiments, the light source 124 can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths and energy levels that can be employed to achieve plasma in the balloon fluid 132 of the catheters 102 described herein. In various embodiments, the pulse widths can include those falling within a range including from at least ten ns to 3000 ns. In some embodiments, the pulse widths can include those falling within a range including from at least 20 ns to 100 ns. In other embodiments, the pulse widths can include those falling within a range including from at least one ns to 500 ns.

Additionally, exemplary nanosecond lasers can include those within the UV to IR spectrum, spanning wavelengths of about ten nanometers (nm) to one millimeter (mm). In some embodiments, the light sources 124 suitable for use in the catheter systems 100 herein can include those capable of producing light at wavelengths of from at least 750 nm to 2000 nm. In other embodiments, the light sources 124 can include those capable of producing light at wavelengths of from at least 700 nm to 3000 nm. In still other embodiments, the light sources 124 can include those capable of producing light at wavelengths of from at least 100 nm to ten micrometers (μm). Nanosecond lasers can include those having repetition rates of up to 200 kHz. In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

The catheter systems 100 disclosed herein can generate pressure waves having maximum pressures in the range of at least one megapascal (MPa) to 100 MPa. The maximum pressure generated by a particular catheter system 100 will depend on the light source 124, the absorbing material, the bubble expansion, the propagation medium, the balloon material, and other factors. In some embodiments, the catheter systems 100 herein can generate pressure waves having maximum pressures in the range of at least two MPa to 50 MPa. In other embodiments, the catheter systems 100 herein can generate pressure waves having maximum pressures in the range of at least two MPa to 30 MPa. In yet other embodiments, the catheter systems 100 herein can generate pressure waves having maximum pressures in the range of at least 15 MPa to 25 MPa.

The pressure waves described herein can be imparted upon the treatment site 106 from a distance within a range from at least 0.1 millimeters (mm) to 25 mm extending radially from the light guides 122A when the catheter 102 is placed at the treatment site 106. In some embodiments, the pressure waves can be imparted upon the treatment site 106 from a distance within a range from at least ten mm to 20 mm extending radially from the light guides 122A when the catheter 102 is placed at the treatment site 106. In other embodiments, the pressure waves can be imparted upon the treatment site 106 from a distance within a range from at least one mm to ten mm extending radially from the light guides 122A when the catheter 102 is placed at the treatment site 106. In yet other embodiments, the pressure waves can be imparted upon the treatment site 106 from a distance within a range from at least 1.5 mm to four mm extending radially from the light guides 122A when the catheter 102 is placed at the treatment site 106. In some embodiments, the pressure waves can be imparted upon the treatment site 106 from a range of at least two MPa to 30 MPa at a distance from 0.1 mm to ten mm. In some embodiments, the pressure waves can be imparted upon the treatment site 106 from a range of at least two MPa to 25 MPa at a distance from 0.1 mm to ten mm.

The power source 125 is electrically coupled to and is configured to provide necessary power to each of the light source 124, the system controller 126, the GUI 127, the handle assembly 128, and the tissue identification system 142. The power source 125 can have any suitable design for such purposes.

As noted, the system controller 126 is electrically coupled to and receives power from the power source 125. Additionally, the system controller 126 is coupled to and is configured to control operation of each of the light source 124, the GUI 127 and the tissue identification system 142. The system controller 126 can include one or more processors or circuits for purposes of controlling the operation of at least the light source 124, the GUI 127 and the tissue identification system 142. For example, the system controller 126 can control the light source 124 for generating pulses of light energy as desired, e.g., at any desired firing rate. Additionally, the system controller 126 can control and/or operate in conjunction with the tissue identification system 142 to effectively provide real-time feedback regarding the type, size and location of any tissue at or near the treatment site 106 in order to optimize treatment in real-time. Further, in certain embodiments, the system controller 126 is configured to receive, process and integrate output from the tissue identification system 142 to provide such desired real-time feedback regarding the type, size and location of any tissue at or near the treatment site 106.

Additionally, the system controller 126 can further be configured to control operation of other components of the catheter system 100, e.g., the positioning of the catheter 102 adjacent to the treatment site 106, the inflation of the balloon 104 with the balloon fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 128.

The GUI 127 is accessible by the user or operator of the catheter system 100. Additionally, the GUI 127 is electrically connected to the system controller 126. With such design, the GUI 127 can be used by the user or operator to ensure that the catheter system 100 is employed as desired to impart pressure onto and induce fractures into the vascular lesions at the treatment site 106. Additionally, the GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time, e.g., during use of the catheter system 100. Further, in various embodiments, the GUI 127 can include one or more colors, different sizes, varying brightness, etc., that may act as alerts to the user or operator. Additionally, or in the alternative, the GUI 127 can provide audio data or information to the user or operator. It is appreciated that the specifics of the GUI 127 can vary depending upon the design requirements of the catheter system 100, or the specific needs, specifications and/or desires of the user or operator.

As shown in FIG. 1, the handle assembly 128 can be positioned at or near the proximal portion 114 of the catheter system 100, and/or near the source manifold 136. Additionally, in this embodiment, the handle assembly 128 is coupled to the balloon 104 and is positioned spaced apart from the balloon 104. Alternatively, the handle assembly 128 can be positioned at another suitable location.

The handle assembly 128 is handled and used by the user or operator to operate, position and control the catheter 102. The design and specific features of the handle assembly 128 can vary to suit the design requirements of the catheter system 100. In the embodiment illustrated in FIG. 1, the handle assembly 128 is separate from, but in electrical and/or fluid communication with one or more of the system controller 126, the light source 124, the fluid pump 138, the GUI 127 and the tissue identification system 142. In some embodiments, the handle assembly 128 can integrate and/or include at least a portion of the system controller 126 within an interior of the handle assembly 128. For example, as shown, in certain such embodiments, the handle assembly 128 can include circuitry 156 that can form at least a portion of the system controller 126. Additionally, in some embodiments, the circuitry 156 can receive electrical signals or data from the tissue identification system 142. Further, or in the alternative, the circuitry 156 can transmit such electrical signals or otherwise provide data to the system controller 126.

In one embodiment, the circuitry 156 can include a printed circuit board having one or more integrated circuits, or any other suitable circuitry. In an alternative embodiment, the circuitry 156 can be omitted, or can be included within the system controller 126, which in various embodiments can be positioned outside of the handle assembly 128, e.g., within the system console 123. It is understood that the handle assembly 128 can include fewer or additional components than those specifically illustrated and described herein.

As noted above, and as provided in greater detail herein below, the tissue identification system 142 is configured to utilize acoustic tissue identification and analysis to effectively provide real-time feedback regarding the type, size, quantity and location of any tissue at or near the treatment site 106 in order to optimize treatment in real-time. Additionally, it is further appreciated that the tissue identification system 142 can have any suitable design for purposes of providing the desired real-time feedback regarding the type, size, quantity and location of any tissue at or near the treatment site 106 in order to optimize treatment in real-time. For example, as described herein below, in various embodiments, the tissue identification system 142 can include at least an acoustic source that converts energy, e.g., light energy and/or electrical energy, into acoustic energy or acoustic waves within the balloon fluid 132 that are directed toward the tissue present at the treatment site 106, and an acoustic detector (also sometimes referred to herein as an “acoustic sensor” or an “acoustic receiver”) that detects, senses and/or receives the acoustic energy after the acoustic energy has impinged upon the tissue present at the treatment site 106. Certain non-exclusive examples of potential designs for the tissue identification system 142 are described in detail herein below.

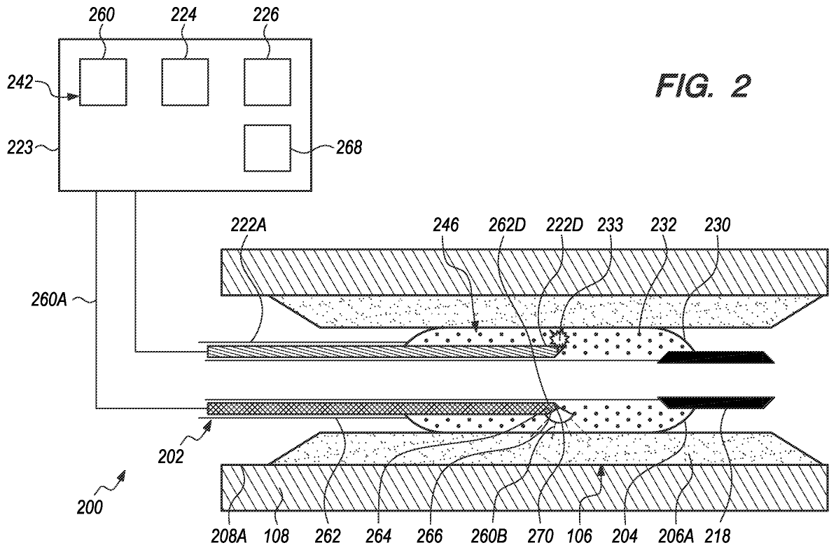

FIG. 2 is a simplified schematic view of a portion of an embodiment of the catheter system 200 including an embodiment of the tissue identification system 242. The design of the catheter system 200 is substantially similar to the embodiments illustrated and described herein above. It is appreciated that various components of the catheter system 200, such as are shown in FIG. 1, are not illustrated in FIG. 2 for purposes of clarity and ease of illustration. However, it is appreciated that the catheter system 200 will likely include most, if not all, of such components, and that such components will be substantially similar in design and function as described in detail herein above.

In various embodiments, as shown in FIG. 2, the catheter system 200 can include a catheter 202 including a balloon 204 having a balloon wall 230 that defines a balloon interior 246, a balloon fluid 232 that is retained substantially within the balloon interior 246, and a guidewire lumen 218 that extends into and runs through the balloon interior 246; an energy source 224, e.g., a light source or other suitable energy source; and one or more energy guides 222A, e.g., light guides or other suitable energy guides. As above, the energy guides 222A are configured to guide energy from the energy source 224 into the balloon interior 246 to generate plasma within the balloon fluid 232, e.g., with a plasma generator 233, at or near a guide distal end 222D of the energy guide 222A disposed within the balloon interior 246 of the balloon 204, which can be located at a treatment site 106 including a vascular lesion 206A within and/or adjacent to a vessel wall 208A of a blood vessel 108 or a heart valve. Further, as above, the plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid 232 retained within the balloon 204 and thereby impart pressure waves upon the treatment site 106.

Additionally, as noted, FIG. 2 also shows an embodiment of the tissue identification system 242 that is configured to utilize acoustic energy to identify the type, size, quantity and location of any tissue at or near the treatment site 106 in order to optimize treatment in real-time. The tissue identification system 242 can have any suitable configuration pursuant to the requirements of the catheter system 200. In certain embodiments, as shown in FIG. 2, the tissue identification system 242 can include one or more of an identification energy source 260, one or more identification energy guides 262 (one is shown in FIG. 2), an acoustic source 264 (also sometimes referred to as an “acoustic transmitter”), an acoustic detector 266 (also sometimes referred to as an “acoustic sensor” or an “acoustic receiver”), and control electronics 268. Alternatively, the tissue identification system 242 can include more components or fewer components than those specifically illustrated and described in relation to FIG. 2.

It is appreciated that although, in various embodiments, the tissue identification systems illustrated herein are described as including an identification light source and one or more identification light guides, the tissue identification systems can alternatively include any suitable identification energy source and identification energy guides for purposes of acoustically interrogating the tissue at the treatment site 106. It is further appreciated that the specific type of identification energy source 260 and identification energy guides 262 will depend upon the specific design of other components of the tissue identification system 242, e.g., the acoustic source 264 and/or the acoustic detector 266.

As illustrated, the tissue identification system 242 will typically include the identification energy source 260 and the identification energy guide(s) 262 that are separate from the energy source 224 and the energy guides 222A that are utilized for generating plasma in the balloon fluid 232 within the balloon interior 246 for purposes of imparting pressure waves upon the treatment site 106. However, in certain alternative embodiments, the tissue identification system can utilize the same energy source 224 and/or the same energy guide(s) 222A that are utilized for generating plasma in the balloon fluid 232 within the balloon interior 246 for purposes of imparting pressure waves upon the treatment site 106. In such alternative embodiments, rather than a very controllable sound source, the energy guide 222A would be used to generate small pressure waves and the reflection of these waves could be acoustically analyzed in a manner similar to what is described in detail herein.

The identification energy source 260 is configured to provide energy, e.g., electrical energy and/or light energy, in the form of an identification source beam 260A that is converted to acoustic energy by the acoustic source 264 and that is directed to impinge upon the tissue of interest, e.g., within the vascular lesion 206A at the treatment site 106. The tissue identification system 242 can utilize any suitable type of identification energy source 260. For example, in various embodiments, the identification energy source 260 can be substantially similar in design and operation to the energy source 124 illustrated and described in detail herein above. Alternatively, the identification energy source 260 can have another suitable design.

As described herein, it is appreciated that certain features and components of the tissue identification system 242 can be modified depending upon the specific design of the tissue identification system 242 and/or depending upon the specific type(s) of tissue that are desired to be identified with the tissue identification system 242. For example, in some embodiments, the tissue identification system 242 can be an ultrasound system, with a pulse echo generator receiver and a small piezoelectric transducer to send and receive acoustic (sound) waves. In one such embodiment, the tissue identification system 242 can utilize intravascular ultrasound (IVUS) that is tailored to calcium or partial calcium (or whatever the specific tissues of interest may be). In such embodiment, a separate electric generator is required for such an electrically-driven tissue identification system. Alternatively, the tissue identification system 242 can be an all-optical acoustic system. In such system, a photoacoustic transducer driven with a laser/light source could generate sound waves and a separate fiber optic with Fabry-Perot cavity can be used to receive the acoustic (sound) waves.

Additionally, the acoustic detector 266 and/or the control electronics 268 can be varied to suit the specific design of the tissue identification system 242. For example, the acoustic detector 266 and/or the control electronics 268 can have a particular design and/or mode of operation (e.g., the control electronics 268 can employ an algorithm of a certain specified design) when the tissue identification system 242 is tailored to identify calcium or partial calcium at the treatment site 106. Alternatively, the acoustic detector 266 and/or the control electronics 268 can have a different design and/or mode of operation (e.g., the control electronics 268 can employ an algorithm having a different specified design) when the tissue identification system 242 is tailored to identify different tissue types at the treatment site 106.

As shown in FIG. 2, the identification source beam 260A from the identification energy source 260 can be directed and/or focused toward and coupled into the identification energy guide 262. The identification energy guide 262 then guides the identification source beam 260A toward the acoustic source 264 that can be positioned at or near and/or be in optical communication with a guide distal end 262D of the identification energy guide 262. The tissue identification system 242 can utilize any suitable type of identification energy guides 262. For example, in various embodiments, the identification energy guides 262 can be substantially similar in design and operation to the energy guides 122A illustrated and described in detail herein above. Alternatively, the identification energy guides 262 can have another suitable design.

The acoustic source 264 is configured to convert the energy of the identification source beam 260A into acoustic energy 260B (illustrated with a series of dashed lines), e.g., acoustic waves, that is directed toward the treatment site 106 including the vascular lesion 206A within and/or adjacent to the vessel wall 208A of the blood vessel 108, or a heart valve. In certain embodiments, the identification energy guide 262 can include a diverter 270 that is positioned at or near the guide distal end 262D of the identification energy guide 262 and that is configured to more accurately and precisely direct the acoustic energy 260B toward the tissue of interest, e.g., the vascular lesion 206A that is present at the treatment site 106 within and/or adjacent to the vessel wall 208A of the blood vessel 108 or the heart valve.

The acoustic source 264 can have any suitable design for purposes of converting the energy of the identification source beam 260A into the desired acoustic energy 260B that is directed toward the tissue of interest at the treatment site 106. For example, in one embodiment, as shown in FIG. 2, the acoustic source 264 can include a piezoelectric transducer that converts the electrical energy of the identification source beam 260A into the desired acoustic energy 260B. Alternatively, the acoustic source 264 can include a photoacoustic transducer that converts the light energy of the identification source beam into the desired acoustic energy, or another suitable acoustic source.

As illustrated, the acoustic energy 260B is directed toward and impinges upon the tissue, e.g., the vascular lesion 206A, located at the treatment site 106. The acoustic energy 260B is subsequently reflected and/or redirected toward the acoustic detector 266 that can be coupled and/or positioned at or near the guide distal end 262D of one of the identification energy guides 262. The acoustic detector 266 is specifically configured to detect and/or sense the acoustic energy 260B (acoustic waves) that have impinged upon the tissue, e.g., the vascular lesion 206A, located at the treatment site 106. More particularly, the acoustic detector 266 can be designed and/or programmed to listen for and identify particular sound signatures from such acoustic energy 260B that are associated with particular tissue types of interest.

The acoustic detector 266 can have any suitable design for purposes of detecting and/or sensing the acoustic energy 260B (acoustic waves) that have impinged upon the tissue, e.g., the vascular lesion 206A, located at the treatment site 106. For example, in certain embodiments, the acoustic detector 266 can include a piezoelectric transducer that detects and/or senses the acoustic energy 260B as desired. In one such embodiment, the piezoelectric transducer of the acoustic detector 266 can be the same piezoelectric transducer of the acoustic source 264. Stated in another manner, in such embodiment, the piezoelectric transducer can function as both the acoustic source 264 and the acoustic detector 266. It is appreciated that the identification energy guide 262 usable with the piezoelectric transducer can have any suitable design and/or can include electrical wires, a converter of electrical signal from the piezoelectric transducer to optical, and/or other suitable designs.

Additionally, or in the alternative, the tissue identification system 242 can employ multiple piezoelectric transducers, i.e. as the acoustic source 264 and/or as the acoustic detector 266, in the catheter 202 for increased spatial resolution. Still alternatively, the acoustic detector 266 can have another suitable design. For example, in one non-exclusive alternative embodiment, the acoustic detector 266 can include a Fabry-Perot cavity that can be included on a separate identification energy guide 262. It is appreciated that other fiber-based sensors can also be utilized as the acoustic detector 266.

Further, it is also appreciated that the acoustic detector 266 can be positioned in any suitable manner within the tissue identification system 242 and/or the catheter system 200. For example, in various embodiments, such as shown in FIG. 2, the acoustic detector 266 can be positioned at or near the guide distal end 262D of an identification energy guide 262. Alternatively, in other embodiments, the acoustic detector 266 can be positioned in any suitable location outside the body 107 (illustrated in FIG. 1) of the patient 109 (illustrated in FIG. 1). In one such alternative embodiment, the acoustic detector 266 can be located on and/or adjacent to the patient 109 in a desirable area to maximize the efficiency of the sound signal. For example, the acoustic detector 266 may be positioned on or underneath the sterile barrier (drape). Alternatively, the acoustic detector 266 can be positioned in another suitable manner to effectively monitor the acoustic energy in the balloon fluid 232 within the balloon interior 246 that is reflected and/or redirected from impingement upon the tissue at the treatment site 106. For example, in certain non-exclusive alternative embodiments, the acoustic detector 266 can be positioned inside and/or adjacent to the system console 223, adjacent to the system controller 226, inside and/or adjacent to the handle assembly 128 (illustrated in FIG. 1), or in another suitable location.

Additionally, the acoustic detector 266 can be electrically coupled to the control electronics 268, i.e. with a wired connection and/or with a wireless connection, for real-time signal measurement. The acoustic detector 266 can generate and provide a detector signal to the control electronics 268, which would be based on the acoustic energy received by the acoustic detector 266 after being reflected back from the tissue at the treatment site 106. The control electronics 268 could then condition the detector signal from the acoustic detector 266 to look for the specific and unique predetermined acoustic identifiers that are associated with the particular tissue types of interest. The control electronics 268 will thus be able to utilize a specially-designed algorithm to effectively and accurately identify the type, size, quantity and location of any tissue at or near the treatment site 106 in order to optimize treatment in real-time. Additionally, the control electronics 268 can then use the information regarding the identity of the type, size, quantity and location of any tissue at or near the treatment site 106 to control at least certain aspects of the catheter system 200, e.g., whether to start treatment, whether to continue treatment, and/or whether to stop treatment based on the status of the tissue at the treatment site at any given time.

It is appreciated that in certain embodiments, the control electronics 268 can form a portion of the system controller 226. Alternatively, the control electronics 268 can be provided separately from the system controller 226 and can be in electrical communication with the system controller 226.

Additionally, it is further appreciated that the tissue identification system 242 can be utilized at any desired time(s) during use of the catheter system 200 for purposes of effectively identifying the type, size, quantity and location of any tissue at or near the treatment site 106. For example, the tissue identification system 242 can be utilized prior to the catheter system 200 being used to generate plasma in the balloon fluid 232 within the balloon interior 246 for purposes of imparting pressure waves upon the treatment site 106. At this point, the tissue identification system 242 can provide evidence of a starting point as far as tissue type, size, quantity and location at the treatment site 106 prior to any treatments being performed. Additionally, the tissue identification system 242 can also be utilized after one or more treatments have been performed, i.e. after the catheter system 200 has been used to generate plasma in the balloon fluid 232 within the balloon interior 246 for purposes of imparting pressure waves upon the treatment site 106, in order to effectively monitor the progress and/or the efficacy of such treatments. Further, the tissue identification system 242 can also be utilized after numerous treatments have been performed in order to confirm the efficacy of such treatments and the elimination of undesired tissue types, e.g., calcified vascular lesions, at the treatment site 106.

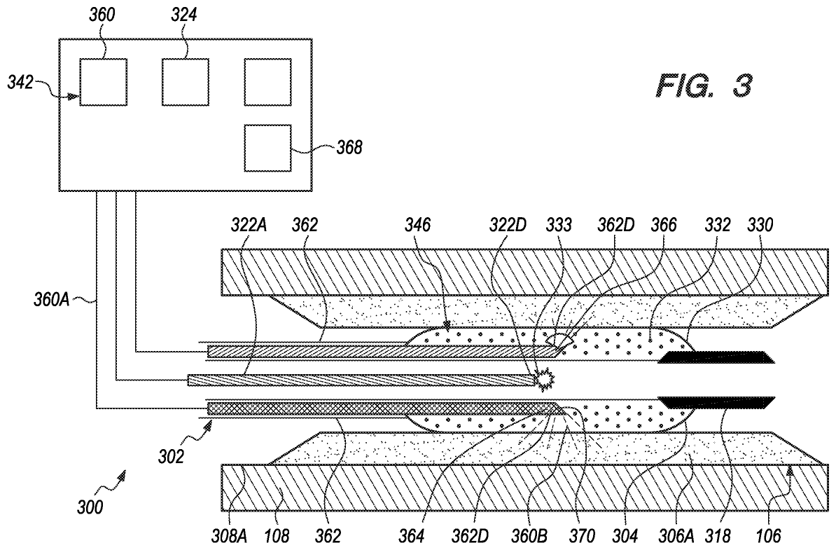

FIG. 3 is a simplified schematic view of a portion of another embodiment of the catheter system 300 including another embodiment of the tissue identification system 342. The design of the catheter system 300 is substantially similar to the embodiments illustrated and described herein above. It is appreciated that various components of the catheter system 300, such as are shown in FIG. 1, are not illustrated in FIG. 3 for purposes of clarity and ease of illustration. However, it is appreciated that the catheter system 300 will likely include most, if not all, of such components, and that such components will be substantially similar in design and function as described in detail herein above.

As shown in FIG. 3, the catheter system 300 can again include a catheter 302 including a balloon 304 having a balloon wall 330 that defines a balloon interior 346, a balloon fluid 332 that is retained substantially within the balloon interior 346, and a guidewire lumen 318 that extends into and runs through the balloon interior 346; an energy source 324, e.g., a light source or other suitable energy source; and one or more energy guides 322A, e.g., light guides or other suitable energy guides. As above, the energy guides 322A are configured to guide energy from the energy source 324 into the balloon interior 346 to generate plasma within the balloon fluid 332, e.g., with a plasma generator 333, at or near a guide distal end 322D of the energy guide 322A disposed within the balloon interior 346 of the balloon 304, which can be located at a treatment site 106 including a vascular lesion 306A within and/or adjacent to a vessel wall 308A of a blood vessel 108, or at a heart valve. Further, as above, the plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid 332 retained within the balloon 304 and thereby impart pressure waves upon the treatment site 106.

Additionally, as noted, FIG. 3 also shows an embodiment of the tissue identification system 342 that is configured to utilize acoustic energy to identify the type, size, quantity and location of any tissue at or near the treatment site 106 in order to optimize treatment in real-time. However, in FIG. 3, the tissue identification system 342 is somewhat different than in the embodiment illustrated in FIG. 2. As illustrated, the tissue identification system 342 can include one or more of an identification energy source 360, e.g., an identification light source, one or more identification energy guides 362 (two are shown in FIG. 3), e.g., one or more identification light guides, an acoustic source 364, an acoustic detector 366, and control electronics 368. Alternatively, the tissue identification system 342 can include more components or fewer components than those specifically illustrated and described in relation to FIG. 3.

In this embodiment, the identification light source 360 is configured to provide light energy in the form of an identification source beam 360A that is converted to acoustic energy by the acoustic source 364 and that is directed to impinge upon the tissue of interest, e.g., within the vascular lesion 306A at the treatment site 106. The tissue identification system 342 can utilize any suitable type of identification light source 360.

As shown in FIG. 3, the identification source beam 360A from the identification light source 360 can be directed and/or focused toward and coupled into an identification light guide 362. The identification light guide 362 then guides the identification source beam 360A toward the acoustic source 364 that can be positioned at or near or in optical communication with a guide distal end 362D of the identification light guide 362.

As shown in this embodiment, the acoustic source 364 is configured to convert the light energy of the identification source beam 360A into acoustic energy 360B (illustrated with a series of dashed lines), e.g., acoustic waves, that is directed toward the treatment site 106 including the vascular lesion 306A within and/or adjacent to the vessel wall 308A of the blood vessel 108, or at a heart valve. In certain embodiments, the identification light guide 362 can include a diverter 370 that is positioned at or near the guide distal end 362D of the identification light guide 362 and that is configured to more accurately and precisely direct the acoustic energy 360B toward the tissue of interest, e.g., the vascular lesion 306A that is present at the treatment site 106.

In this embodiment, the acoustic source 364 includes a photoacoustic transducer that converts the light energy of the identification source beam 360A into the desired acoustic energy 360B that is directed toward the tissue of interest at the treatment site 106. Additionally, as illustrated, the acoustic energy 360B is directed toward and impinges upon the tissue, e.g., the vascular lesion 306A, located at the treatment site 106. The acoustic energy 360B is subsequently reflected and/or redirected toward the acoustic detector 366 that is coupled and/or positioned at or near the guide distal end 362D of another of the identification energy guides 362.

Further, the acoustic detector 366 is again configured to detect and/or sense the acoustic energy 360B (acoustic waves) that have impinged upon the tissue, e.g., the vascular lesion 306A, located at the treatment site 106. However, in this embodiment, the acoustic detector 366 includes a Fabry-Perot cavity that can be included on a separate identification energy guide 362.

As above, it is appreciated that the tissue identification system 342 can employ multiple acoustic sources 364 and/or multiple acoustic detectors 366 in the catheter 302 for increased spatial resolution.

Additionally, the acoustic detector 366 can again be electrically coupled to the control electronics 368, i.e. with a wired connection and/or with a wireless connection, for real-time signal measurement. The acoustic detector 366 can generate and provide a detector signal to the control electronics 368, which would be based on the acoustic energy received by the acoustic detector 366 after being reflected back from the tissue at the treatment site 106. The control electronics 368 could then condition the detector signal from the acoustic detector 366 to look for the specific and unique predetermined acoustic identifiers that are associated with the particular tissue types of interest. The control electronics 368 will thus be able to utilize a specially-designed algorithm to effectively and accurately identify the type, size, quantity and location of any tissue at or near the treatment site 106 in order to optimize treatment in real-time.

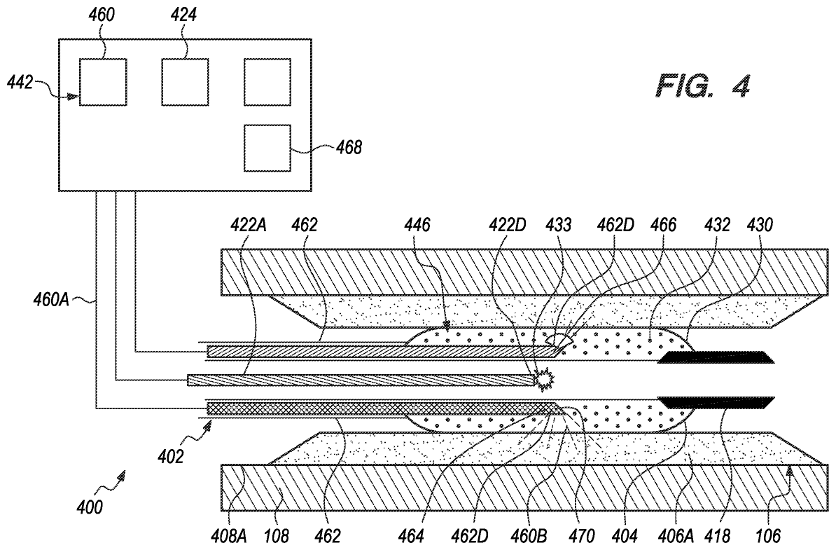

FIG. 4 is a simplified schematic view of a portion of still another embodiment of the catheter system 400 including still another embodiment of the tissue identification system 442. The design of the catheter system 400 is substantially similar to the embodiments illustrated and described herein above. It is appreciated that various components of the catheter system 400, such as are shown in FIG. 1, are not illustrated in FIG. 4 for purposes of clarity and ease of illustration. However, it is appreciated that the catheter system 400 will likely include most, if not all, of such components, and that such components will be substantially similar in design and function as described in detail herein above.

As shown in FIG. 4, the catheter system 400 can again include a catheter 402 including a balloon 404 having a balloon wall 430 that defines a balloon interior 446, a balloon fluid 432 that is retained substantially within the balloon interior 446, and a guidewire lumen 418 that extends into and runs through the balloon interior 446; an energy source 424, e.g., a light source or other suitable energy source; and one or more energy guides 422A, e.g., light guides or other suitable energy guides. As above, the energy guides 422A are configured to guide energy from the energy source 424 into the balloon interior 446 to generate plasma within the balloon fluid 432, e.g., with a plasma generator 433, at or near a guide distal end 422D of the energy guide 422A disposed within the balloon interior 446 of the balloon 404, which can be located at a treatment site 106 including a vascular lesion 406A within and/or adjacent to a vessel wall 408A of a blood vessel 108, or at a heart valve. Further, as above, the plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid 432 retained within the balloon 404 and thereby impart pressure waves upon the treatment site 106.

Additionally, as noted, FIG. 4 also shows an embodiment of the tissue identification system 442 that is configured to utilize acoustic energy to identify the type, size, quantity and location of any tissue at or near the treatment site 106 in order to optimize treatment in real-time. However, in FIG. 4, the tissue identification system 442 is somewhat different than in the previous embodiments. As illustrated, the tissue identification system 442 can include one or more of an identification energy source 460, e.g., an identification light source, one or more identification energy guides 462 (two are shown in FIG. 4), an acoustic source 464, an acoustic detector 466, and control electronics 468. Alternatively, the tissue identification system 442 can include more components or fewer components than those specifically illustrated and described in relation to FIG. 4.

As above, the identification light source 460 is configured to provide light energy in the form of an identification source beam 460A that is converted to acoustic energy by the acoustic source 464 and that is directed to impinge upon the tissue of interest, e.g., within the vascular lesion 406A at the treatment site 106. The tissue identification system 442 can utilize any suitable type of identification light source 460.

As shown in FIG. 4, the identification source beam 460A from the identification light source 460 can be directed and/or focused toward and coupled into an identification light guide 462. The identification light guide 462 then guides the identification source beam 460A toward the acoustic source 464 that can be positioned at or near or in optical communication with a guide distal end 462D of the identification light guide 462.

As with the previous embodiment, the acoustic source 464 is configured to convert the light energy of the identification source beam 460A into acoustic energy 460B (illustrated with a series of dashed lines), e.g., acoustic waves, that is directed toward the treatment site 106 including the vascular lesion 406A. Additionally, as illustrated, in certain embodiments, the identification light guide 462 can include a diverter 470 that is positioned at or near the guide distal end 462D of the identification light guide 462 and that is configured to more accurately and precisely direct the acoustic energy 460B toward the tissue of interest, e.g., the vascular lesion 406A that is present at the treatment site 106.

In this embodiment, the acoustic source 464 again includes a photoacoustic transducer that converts the light energy of the identification source beam 460A into the desired acoustic energy 460B that is directed toward the tissue of interest at the treatment site 106. Additionally, as illustrated, the acoustic energy 460B is directed toward and impinges upon the tissue, e.g., the vascular lesion 406A, located at the treatment site 106. The acoustic energy 460B is subsequently reflected and/or redirected toward the acoustic detector 466 that is coupled and/or positioned at or near the guide distal end 462D of another of the identification energy guides 462.

Further, the acoustic detector 466 is again configured to detect and/or sense the acoustic energy 460B (acoustic waves) that have impinged upon the tissue, e.g., the vascular lesion 406A, located at the treatment site 106. In this embodiment, the acoustic detector 466 includes a piezoelectric transducer that can be included on a separate identification energy guide 462. It is appreciated that the identification energy guide 462 usable with the piezoelectric transducer can have any suitable design and/or can include electrical wires, a converter of electrical signal from the piezoelectric transducer to optical, and/or other suitable designs.

As above, it is appreciated that the tissue identification system 442 can employ multiple acoustic sources 464 and/or multiple acoustic detectors 466 in the catheter 402 for increased spatial resolution.

Additionally, the acoustic detector 466 can again be electrically coupled to the control electronics 468, i.e. with a wired connection and/or with a wireless connection, for real-time signal measurement. The acoustic detector 466 can generate and provide a detector signal to the control electronics 468, which would be based on the acoustic energy received by the acoustic detector 466 after being reflected back from the tissue at the treatment site 106. The control electronics 468 could then condition the detector signal from the acoustic detector 466 to look for the specific and unique predetermined acoustic identifiers that are associated with the particular tissue types of interest. The control electronics 468 will thus be able to utilize a specially-designed algorithm to effectively and accurately identify the type, size, quantity and location of any tissue at or near the treatment site 106 in order to optimize treatment in real-time.

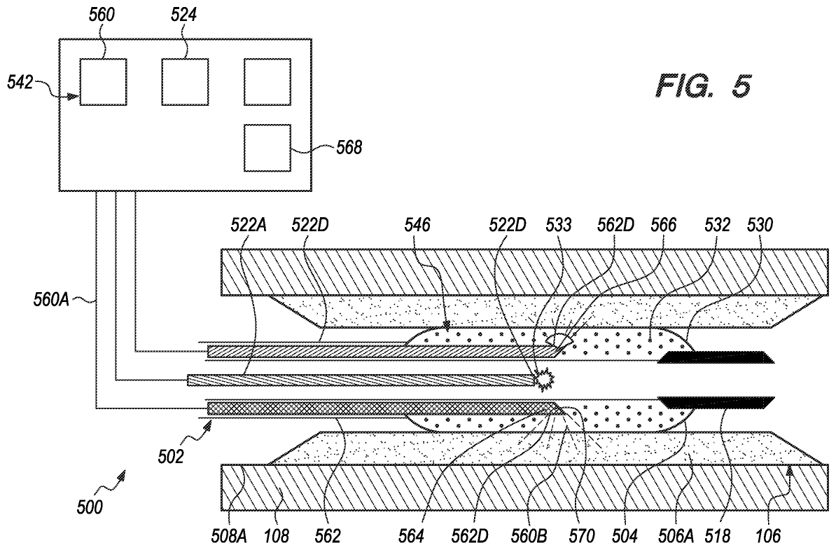

FIG. 5 is a simplified schematic view of a portion of yet another embodiment of the catheter system 500 including yet another embodiment of the tissue identification system 542. The design of the catheter system 500 is substantially similar to the embodiments illustrated and described herein above. It is appreciated that various components of the catheter system 500, such as are shown in FIG. 1, are not illustrated in FIG. 5 for purposes of clarity and ease of illustration. However, it is appreciated that the catheter system 500 will likely include most, if not all, of such components, and that such components will be substantially similar in design and function as described in detail herein above.

As shown in FIG. 5, the catheter system 500 can again include a catheter 502 including a balloon 504 having a balloon wall 530 that defines a balloon interior 546, a balloon fluid 532 that is retained substantially within the balloon interior 546, and a guidewire lumen 518 that extends into and runs through the balloon interior 546; an energy source 524, e.g., a light source or other suitable energy source; and one or more energy guides 522A, e.g., light guides or other suitable energy guides. As above, the energy guides 522A are configured to guide energy from the energy source 524 into the balloon interior 546 to generate plasma within the balloon fluid 532, e.g., with a plasma generator 533, at or near a guide distal end 522D of the energy guide 522A disposed within the balloon interior 546 of the balloon 504, which can be located at a treatment site 106 including a vascular lesion 506A within and/or adjacent to a vessel wall 508A of a blood vessel 108, or at a heart valve. Further, as above, the plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid 532 retained within the balloon 504 and thereby impart pressure waves upon the treatment site 106.

Additionally, as noted, FIG. 5 also shows an embodiment of the tissue identification system 542 that is configured to utilize acoustic energy to identify the type, size, quantity and location of any tissue at or near the treatment site 106 in order to optimize treatment in real-time. However, in FIG. 5, the tissue identification system 542 is somewhat different than in the previous embodiments. As illustrated, the tissue identification system 542 can include one or more of an identification energy source 560, one or more identification energy guides 562 (two are shown in FIG. 5), an acoustic source 564, an acoustic detector 566, and control electronics 568. Alternatively, the tissue identification system 542 can include more components or fewer components than those specifically illustrated and described in relation to FIG. 5.

As above, the identification energy source 560 is configured to provide energy in the form of an identification source beam 560A that is converted to acoustic energy by the acoustic source 564 and that is directed to impinge upon the tissue of interest, e.g., within the vascular lesion 506A at the treatment site 106. The tissue identification system 542 can utilize any suitable type of identification energy source 560.

As shown in FIG. 5, the identification source beam 560A from the identification energy source 560 can be directed and/or focused toward and coupled into an identification energy guide 562. The identification energy guide 562 then guides the identification source beam 560A toward the acoustic source 564 that can be positioned at or near or in optical communication with a guide distal end 562D of the identification energy guide 562.

As with the previous embodiments, the acoustic source 564 is configured to convert the energy of the identification source beam 560A into acoustic energy 560B (illustrated with a series of dashed lines), e.g., acoustic waves, that is directed toward the treatment site 106 including the vascular lesion 506A. Additionally, as illustrated, in certain embodiments, the identification energy guide 562 can include a diverter 570 that is positioned at or near the guide distal end 562D of the identification energy guide 562 and that is configured to more accurately and precisely direct the acoustic energy 560B toward the tissue of interest, e.g., the vascular lesion 506A that is present at the treatment site 106.

In this embodiment, the acoustic source 564 again includes a piezoelectric transducer that converts the electrical energy of the identification source beam 560A into the desired acoustic energy 560B that is directed toward the tissue of interest at the treatment site 106. Additionally, as illustrated, the acoustic energy 560B is directed toward and impinges upon the tissue, e.g., the vascular lesion 506A, located at the treatment site 106. The acoustic energy 560B is subsequently reflected and/or redirected toward the acoustic detector 566 that is coupled and/or positioned at or near the guide distal end 562D of another of the identification energy guides 562.

Further, the acoustic detector 566 is again configured to detect and/or sense the acoustic energy 560B (acoustic waves) that have impinged upon the tissue, e.g., the vascular lesion 506A, located at the treatment site 106. In this embodiment, the acoustic detector 566 again includes a Fabry-Perot cavity that can be included on a separate identification energy guide 562.

As above, it is appreciated that the tissue identification system 542 can employ multiple acoustic sources 564 and/or multiple acoustic detectors 566 in the catheter 502 for increased spatial resolution.

Additionally, the acoustic detector 566 can again be electrically coupled to the control electronics 568, i.e. with a wired connection and/or with a wireless connection, for real-time signal measurement. The acoustic detector 566 can generate and provide a detector signal to the control electronics 568, which would be based on the acoustic energy received by the acoustic detector 566 after being reflected back from the tissue at the treatment site 106. The control electronics 568 could then condition the detector signal from the acoustic detector 566 to look for the specific and unique predetermined acoustic identifiers that are associated with the particular tissue types of interest. The control electronics 568 will thus be able to utilize a specially-designed algorithm to effectively and accurately identify the type, size, quantity and location of any tissue at or near the treatment site 106 in order to optimize treatment in real-time.

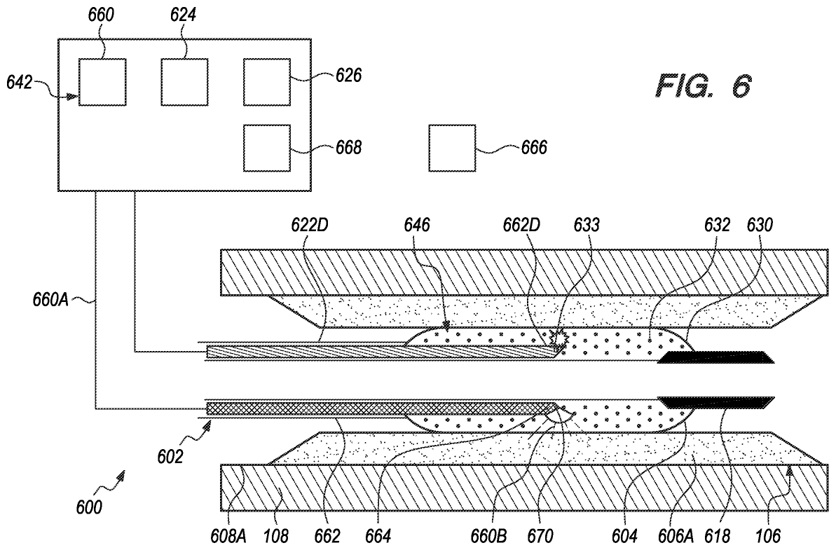

FIG. 6 is a simplified schematic view of a portion of still yet another embodiment of the catheter system 600 including still yet another embodiment of the tissue identification system 642. The design of the catheter system 600 is substantially similar to the embodiments illustrated and described herein above. It is appreciated that various components of the catheter system 600, such as are shown in FIG. 1, are not illustrated in FIG. 6 for purposes of clarity and ease of illustration. However, it is appreciated that the catheter system 600 will likely include most, if not all, of such components, and that such components will be substantially similar in design and function as described in detail herein above.

As shown in FIG. 6, the catheter system 600 can again include a catheter 602 including a balloon 604 having a balloon wall 630 that defines a balloon interior 646, a balloon fluid 632 that is retained substantially within the balloon interior 646, and a guidewire lumen 618 that extends into and runs through the balloon interior 646; an energy source 624, e.g., a light source or other suitable energy source; and one or more energy guides 622A, e.g., light guides or other suitable energy guides. As above, the energy guides 622A are configured to guide energy from the energy source 624 into the balloon interior 646 to generate plasma within the balloon fluid 632, e.g., with a plasma generator 633, at or near a guide distal end 622D of the energy guide 622A disposed within the balloon interior 646 of the balloon 604, which can be located at a treatment site 106 including a vascular lesion 606A within and/or adjacent to a vessel wall 608A of a blood vessel 108, or at a heart valve. Further, as above, the plasma formation can initiate a pressure wave and can initiate the rapid formation of one or more bubbles that can rapidly expand to a maximum size and then dissipate through a cavitation event that can launch a pressure wave upon collapse. The rapid expansion of the plasma-induced bubbles can generate one or more pressure waves within the balloon fluid 632 retained within the balloon 604 and thereby impart pressure waves upon the treatment site 106.

Additionally, as noted, FIG. 6 also shows an embodiment of the tissue identification system 642 that is configured to utilize acoustic energy to identify the type, size, quantity and location of any tissue at or near the treatment site 106 in order to optimize treatment in real-time. However, in FIG. 6, the tissue identification system is somewhat different than in the previous embodiments. As illustrated, the tissue identification system 642 can include one or more of an identification energy source 660, one or more identification energy guides 662 (one is shown in FIG. 6), an acoustic source 664, an acoustic detector 666, and control electronics 668. Alternatively, the tissue identification system 642 can include more components or fewer components than those specifically illustrated and described in relation to FIG. 6.

As above, the identification energy source 660 is configured to provide energy, e.g., light energy, electrical energy, etc., in the form of an identification source beam 660A that is converted to acoustic energy by the acoustic source 664 and that is directed to impinge upon the tissue of interest, e.g., within the vascular lesion 606A at the treatment site 106. The tissue identification system 642 can utilize any suitable type of identification energy source 660.

As shown in FIG. 6, the identification source beam 660A from the identification energy source 660 can be directed and/or focused toward and coupled into an identification energy guide 662. The identification energy guide 662 then guides the identification source beam 660A toward the acoustic source 664 that can be positioned at or near or in optical communication with a guide distal end 662D of the identification energy guide 662.

As with the previous embodiments, the acoustic source 664 is configured to convert the energy of the identification source beam 660A into acoustic energy 660B (illustrated with a series of dashed lines), e.g., acoustic waves, that is directed toward the treatment site 106 including the vascular lesion 606A. Additionally, as illustrated, in certain embodiments, the identification energy guide 662 can include a diverter 670 that is positioned at or near the guide distal end 662D of the identification energy guide 662 and that is configured to more accurately and precisely direct the acoustic energy 660B toward the tissue of interest, e.g., the vascular lesion 606A that is present at the treatment site 106.

In this embodiment, the acoustic source 664 can include any suitable type of acoustic source, e.g., a piezoelectric transducer, a photoacoustic transducer, or another suitable acoustic source, that converts the light energy or electrical energy of the identification source beam 660A into the desired acoustic energy 660B that is directed toward the tissue of interest at the treatment site 106. Additionally, as illustrated, the acoustic energy 660B is directed toward and impinges upon the tissue, e.g., the vascular lesion 606A, located at the treatment site 106. The acoustic energy 660B is subsequently reflected by the tissue and is subsequently detected and/or sensed by the acoustic detector 666.

However, in this embodiment, the acoustic detector 666 is positioned in a different manner than in the previous embodiments. More particularly, as illustrated in FIG. 6, the acoustic detector 666 is positioned outside the body 107 (illustrated in FIG. 1) of the patient 109 (illustrated in FIG. 1). For example, in one embodiment, the acoustic detector 666 can be located on and/or adjacent to the patient 109, e.g., on or underneath the sterile barrier (drape), in a desirable area to maximize the efficiency of the sound signal. Alternatively, the acoustic detector 666 can be positioned in another suitable manner to effectively monitor the acoustic energy in the balloon fluid 632 within the balloon interior 646 that is reflected and/or redirected from impingement upon the tissue at the treatment site 106. For example, in certain non-exclusive alternative embodiments, the acoustic detector 666 can be positioned inside and/or adjacent to the system console 623, adjacent to the system controller 626, inside and/or adjacent to the handle assembly 128 (illustrated in FIG. 1), or in another suitable location.

Further, the acoustic detector 666 is again configured to detect and/or sense the acoustic energy 660B (acoustic waves) that have impinged upon the tissue, e.g., the vascular lesion 606A, located at the treatment site 106. In alternative embodiments, the acoustic detector 666 can include a piezoelectric transducer, a Fabry-Perot cavity, or another suitable type of acoustic detector.

As above, it is appreciated that the tissue identification system 642 can employ multiple acoustic sources 664 and/or multiple acoustic detectors 666 in the catheter system 600 for increased spatial resolution.

Additionally, the acoustic detector 666 can again be electrically coupled to the control electronics 668, i.e. with a wired connection and/or with a wireless connection, for real-time signal measurement. The acoustic detector 666 can generate and provide a detector signal to the control electronics 668, which would be based on the acoustic energy received by the acoustic detector 666 after being reflected back from the tissue at the treatment site 106. The control electronics 668 could then condition the detector signal from the acoustic detector 666 to look for the specific and unique predetermined acoustic identifiers that are associated with the particular tissue types of interest. The control electronics 668 will thus be able to utilize a specially-designed algorithm to effectively and accurately identify the type, size, quantity and location of any tissue at or near the treatment site 106 in order to optimize treatment in real-time.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the present detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter system and the tissue identification system have been illustrated and described herein, one or more features of any one embodiment can be combined with one or more features of one or more of the other embodiments, provided that such combination satisfies the intent of the present invention.

While a number of exemplary aspects and embodiments of the catheter system and the tissue identification system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

1. A catheter system for treating a treatment site within or adjacent to a vessel wall or a heart valve within a body of a patient, the catheter system comprising:
an energy source that generates energy;
a balloon that is positionable substantially adjacent to the treatment site, the balloon having a balloon wall that defines a balloon interior, the balloon being configured to retain a balloon fluid within the balloon interior;
an energy guide that is configured to receive energy from the energy source and guide the energy into the balloon interior so that a plasma is formed in the balloon fluid within the balloon interior; and
a tissue identification system that is configured to acoustically analyze tissue within the treatment site.

2. The catheter system of claim 1 wherein the tissue identification system is configured to utilize acoustic tissue identification to provide real-time feedback regarding tissue type and quantity within the treatment site.

3. The catheter system of claim 1 wherein the energy source is a laser source that provides pulses of laser energy.

4. The catheter system of claim 3 wherein the energy guide includes an optical fiber.

5. The catheter system of claim 1 wherein the energy source is a high voltage energy source that provides pulses of high voltage energy.

6. The catheter system of claim 5 wherein the energy guide includes an electrode pair having spaced apart electrodes that extend into the balloon interior; and wherein pulses of high voltage energy from the energy source are applied to the electrodes and form an electrical arc across the electrodes.

7. The catheter system of claim 1 wherein the tissue identification system includes (i) an identification energy source that generates energy, and (ii) an acoustic source that receives the energy from the identification energy source in the form of an identification source beam and converts the identification source beam into acoustic energy that is directed toward the tissue within the treatment site.

8. The catheter system of claim 7 wherein the tissue identification system is an ultrasound system, and wherein the identification energy source includes a pulse echo generator.

9. The catheter system of claim 7 wherein the identification energy source includes a light source.

10. The catheter system of claim 7 wherein the identification energy source includes a laser.

11. The catheter system of claim 7 wherein the acoustic source includes a piezoelectric transducer.

12. The catheter system of claim 7 wherein the acoustic source includes a photoacoustic transducer.

13. The catheter system of claim 7 wherein the tissue identification system further includes an identification energy guide that guides the identification source beam from the identification energy source into the balloon interior.

14. The catheter system of claim 7 wherein the tissue identification system further includes an acoustic detector that is configured to detect acoustic energy within the balloon interior.

15. The catheter system of claim 14 wherein at least a portion of the acoustic energy directed toward the treatment site is reflected by the tissue within the treatment site and is directed toward the acoustic detector.

16. The catheter system of claim 14 wherein the acoustic detector is positioned outside the body of the patient.

17. The catheter system of claim 14 wherein the acoustic detector includes a piezoelectric transducer.

18. The catheter system of claim 14 wherein the acoustic detector generates a detector signal based on the detected acoustic energy within the balloon interior and sends the detector signal to control electronics.

19. The catheter system of claim 18 wherein the control electronics analyze the detector signal to determine the tissue type and quantity within the treatment site.

20. A method for treating a treatment site within or adjacent to a vessel wall or heart valve within a body of a patient, the method comprising the steps of:
generating energy with an energy source;
positioning a balloon substantially adjacent to the treatment site, the balloon having a balloon wall that defines a balloon interior;
retaining a balloon fluid within the balloon interior;
receiving energy from the energy source with an energy guide;
guiding the energy with the energy guide into the balloon interior so that plasma is formed in the balloon fluid within the balloon interior; and
acoustically analyzing tissue within the treatment site with a tissue identification system.

21. The catheter system of claim 1 wherein the balloon includes a drug-eluting coating.
